(19) United States
(12) Patent Application Publication
(10) Pub. No.: US 2026/0117304 A1
(43) Pub. Date: Apr. 30, 2026

(54) ANALYSIS METHOD OF SPATIAL TRANSCRIPTOME-BASED 4D-PRINTED STEM CELL SCAFFOLD FOR IMPROVING DIABETIC SKIN INJURY

(71) Applicant: QINGDAO KANGMINGBEI JIAN BIOPHARMACEUTICAL CO., LTD., Qingdao City (CN)

(72) Inventors: Wenhua XU, Qingdao City (CN); Xiaomin WANG, Qingdao City (CN); Yan TANG, Qingdao City (CN); Xiao XU, Qingdao City (CN); Xiaoying KONG, Qingdao City (CN); Mengyu ZHANG, Qingdao City (CN); Jiaxu SONG, Qingdao City (CN); Xin MA, Qingdao City (CN); Junlin LV, Qingdao City (CN); Yiwei XU, Qingdao City (CN); Zhen ShANG, Qingdao City (CN); Nailong PAN, Qingdao City (CN); Liang ZHANG, Qingdao City (CN); Dan HAN, Qingdao City (CN)

(73) Assignee: QINGDAO KANGMINGBEI JIAN BIOPHARMACEUTICAL CO., LTD, Qingdao City, SD (CN)

(21) Appl. No.: 19/216,808

(22) Filed: May 23, 2025

(30) Foreign Application Priority Data
Oct. 24, 2024 (CN) 2024114871213

(51) Int. Cl. C12Q 1/6883; C12Q 1/6869

(52) U.S. Cl. CPC C12Q 1/6883; C12Q 1/6869; C12Q 2600/158

(57) ABSTRACT

The invention relates to bioinformatics technology and specifically to an analysis method of a spatial transcriptome-based 4D-printed stem cell scaffold for enhancing diabetic skin injury healing. Spatial transcriptome sequencing is performed on tissues from both the PBS group and the combined treatment group. Cell clustering and annotation identify main cell types, followed by visualization of their spatial distributions in both groups. The proportions of various cell types are statistically quantified and plotted. Cell subsets exhibiting significant differential gene expression are enriched and verified. Using a 4D-printed stem cell scaffold combined with a piRNA inhibitor and stem cell therapy, the method reveals spatial distribution and dynamic cellular changes during wound healing. It identifies precise cellular locations and gene expression patterns within tissues, providing critical insights into the biological processes by which biomaterials promote diabetic injury repair. This approach offers new directions for future drug development.

### TECHNICAL FIELD

The invention belongs to the field of bioinformatics technology, specifically, it is an analysis method of a spatial transcriptome-based 4D-printed stem cell scaffold for improving diabetic skin injury, and provides a new perspective for the study of the healing mechanism of diabetic skin injury.

### BACKGROUND

Skin injury (foot ulcers and lower limb erosion, etc.) is one of the common complications caused by diabetes, it has the characteristics of a slow healing process and chronic inflammation. During the healing process, immune cells, cytokines, and other components in the local microenvironment of the injury interact to affect the inflammation, proliferation, and remodeling of wound healing. In addition, cell function and signal transduction in the hyperglycemic microenvironment will also be affected, resulting in difficult wound healing.

The 4D-printed stem cell scaffold is a biological material prepared by chitosan and its derivatives, it has the characteristics of efficient transfer of stem cells and can effectively help the wound healing of diabetic skin injury. Although therapeutic effects have been confirmed, it is still lacking to explore the mechanism of its related role. At present, there is a lack of appropriate research methods for the mechanism of biomaterials in repairing diabetic skin injury, it is often limited to the analysis of single cell type or molecular signal, and it is impossible to further study the dynamic changes of local microenvironment, which seriously limits the comprehensive understanding of the healing mechanism of diabetic skin injury. In addition, the research methods in the existing technology have some limitations in analyzing the cell interaction and signal network in the local microenvironment of injury. Based on this, it is necessary to develop new technical means for further research to comprehensively analyze the cellular composition, molecular signals, and dynamic changes of the local microenvironment of diabetic skin injury.

The application of spatial transcriptomics technology in the study of diabetic skin wound healing has become increasingly important, it can reveal the precise location and gene expression pattern of cells in tissues, and provide a new perspective for understanding the complex biological processes occurring in the local microenvironment of diabetic skin injury wounds. Through spatial transcriptomics technology, the spatial distribution of cell types and the dynamic changes of gene expression during wound healing of diabetic skin injury can be described in detail, and the intercellular interaction and signal transduction network during wound healing of diabetic skin injury can be analyzed, it is very important to study the mechanism of slow wound healing of diabetic skin injury and the role of chronic inflammation in the healing process.

Chinese patent 202210106899.X published a method for analyzing the improvement of myocardial fibrosis in atrial fibrillation by Qipo Shengmai composition based on spatial transcriptome technology, including the following steps:

1) The spatial transcriptome technology was used to perform spatial transcriptome sequencing on the myocardial tissue sections of the normal group, the atrial fibrillation model group, and the Qipo Shengmai administration group, and the spatial transcriptome data of the samples in the normal group, the model group, and the Qipo Shengmai administration group were obtained.

2) The sctransform (SCT) algorithm in the Seurat package of the R language was used to standardize the expression matrix data of the spatial transcriptome data conversion of each sample; then, the highly variable genes were screened by the vst algorithm on the standardized data, and the differentially expressed gene sets were obtained.

3) The Single Cell Transformation (SCT) algorithm in the Seurat package of R language was used to standardize the differentially expressed gene sets obtained from each sample; then, the PCA method was used to perform linear dimensionality reduction on the standardized data to obtain PCA dimensionality reduction data. Then, the PCA dimensionality reduction data is processed by the T-SNE nonlinear dimensionality reduction algorithm to obtain T-SNE dimensionality reduction data.

4) Cluster analysis of T-SNE dimensionality reduction data based on the SNN clustering algorithm was carried out to obtain the differential gene cluster subsets (Cluster) of the expression changes of the samples in the normal group, the model group, and the Qipo Shengmai administration group.

5) The wilcox rank sum test of Seurat software was used to compare the genes of each cluster subgroup of the normal group, the model group, and the Qipo Shengmai administration group with the genes of all other cluster subgroups of the corresponding group, and the cluster differential gene set of each cluster was obtained (i.e., the gene set with large expression difference of the cluster).

6) The R language ClusterProfiler package was used to perform differential gene enrichment analysis on the cluster differential gene sets of each cluster of samples in the normal group, the model group, and the Qipo Shengmai administration group, respectively, the results of differential gene enrichment analysis between the normal group and the model group and the Qipo Shengmai administration group were compared, respectively, and the main difference groups of the model group and the Qipo Shengmai administration group were screened.

7) Differential gene function enrichment analysis was performed on the characteristic differential gene sets of the main differential gene groups of the samples of the Qipo Shengmai administration group and the main differential gene groups of the model group samples, and the related biological processes and signaling pathways of the characteristic differential genes of Qipo Shengmai combination intervention and improvement of atrial fibrillation myocardial fibrosis were obtained, it is mainly aimed at atrial fibrillation myocardial fibrosis, which is different from diabetic skin injury, and does not point out the specific pathways and cell types that play a role.

Although spatial transcriptomics technology lacks systematic research methods and systematic analysis strategies in the field of wound healing of diabetic skin injury. Therefore, it is necessary to further develop and optimize spatial transcriptomics technology and related bioinformatics analysis methods to comprehensively and deeply understand the wound healing process of diabetic skin injury, so as to reveal the healing mechanism of biological materials on diabetic skin injury.

### SUMMARY

The purpose of this invention is to overcome the shortcomings of existing technologies, develop and design an analysis method of a 4D-printed stem cell scaffold for improving diabetic skin injury, and reveal the spatial distribution and dynamic changes of cells during wound healing of diabetic skin injury.

In order to achieve the above purpose, the specific process of the analysis method of the spatial transcriptome-based 4D-printed stem cell scaffold for improving diabetic skin injury is as follows:
firstly, the tissues of the phosphate buffered saline solution (PBS) treatment group and the combined treatment group (4D-printed stem cell scaffold combined with piRNA inhibitors and the stem cell treatment regimen) are subjected to spatial transcriptome sequencing to obtain two sets of data;
then, cell clustering and cell annotation are performed, after identifying the main cell types, the spatial distributions of the two groups of cell types are visualized, and the proportions of various cell types are statistically quantified and plotted;
finally, the cell subsets with significant differential genes are enriched and verified.

Compared with the existing technology, this invention provides a research method to study the specific mechanism of biomaterials in the process of wound healing in the back of diabetic mice by using spatial transcriptomics technology, by combining 4D-printed stem cell scaffold with piRNA inhibitors and stem cells, the spatial distribution and dynamic changes of cells during wound healing are demonstrated, and the precise location and gene expression patterns of cells in tissues are revealed. At the same time, Ptpn3-T cells related to wound healing are identified by spatial transcriptome technology, it can provide new ideas for subsequent drug development, both economic and social benefits, and also provide a new perspective for the study of the healing mechanism of diabetic skin injury.

### BRIEF DESCRIPTION OF THE DRAWINGS

FIG. 1. Spatial localization map of different cell types.

FIG. 2. Histogram of the proportion of different cell types.

FIG. 3. Heat map of differential gene in different treatment groups.

FIG. 4. Spatial localization map of the T cell subtypes.

FIG. 5. Histogram of the proportion of T cell subtypes.

FIG. 6. Violin diagram of differentially expressed genes in T cell cluster 0 related to the invention.

FIG. 7. KEGG analysis related to the invention in different treatment groups.

FIG. 8. GO analysis related to the invention in different treatment groups.

### DETAILED DESCRIPTION OF THE EMBODIMENTS

The following is a further explanation of the invention in combination with the attached figures and the specific implementation method.

### Example 1

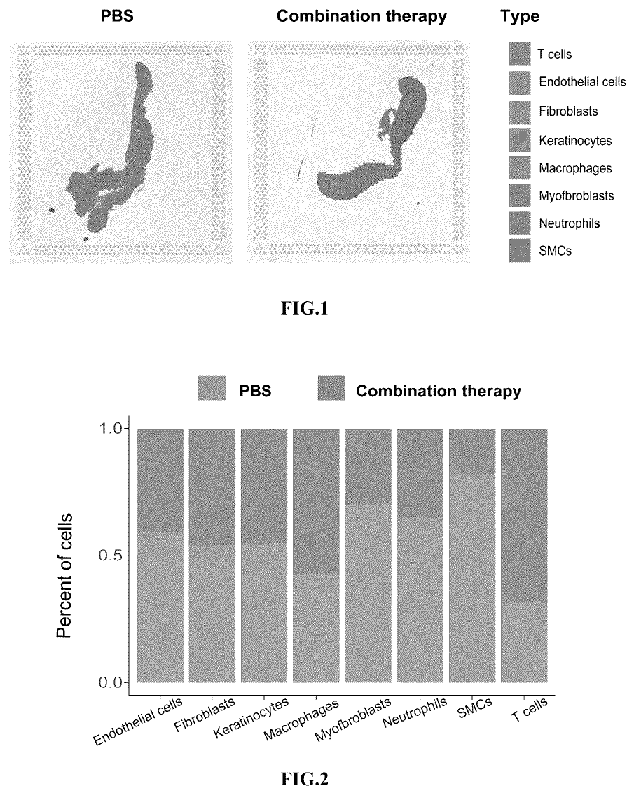

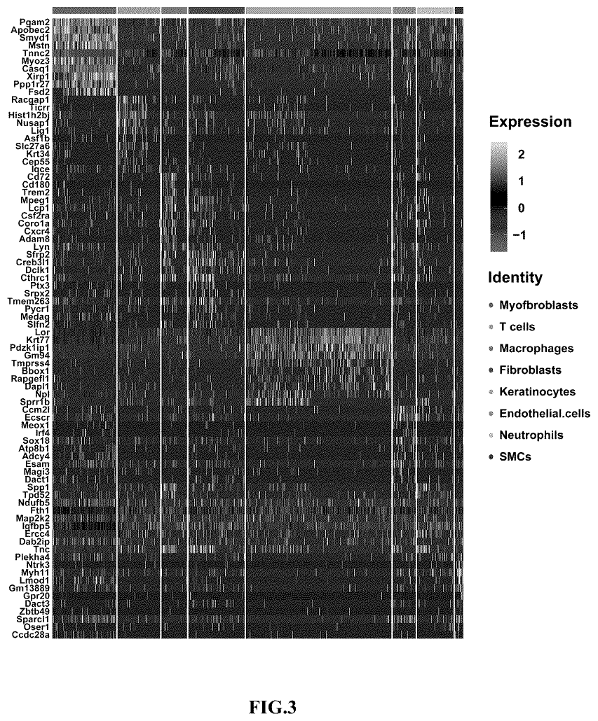

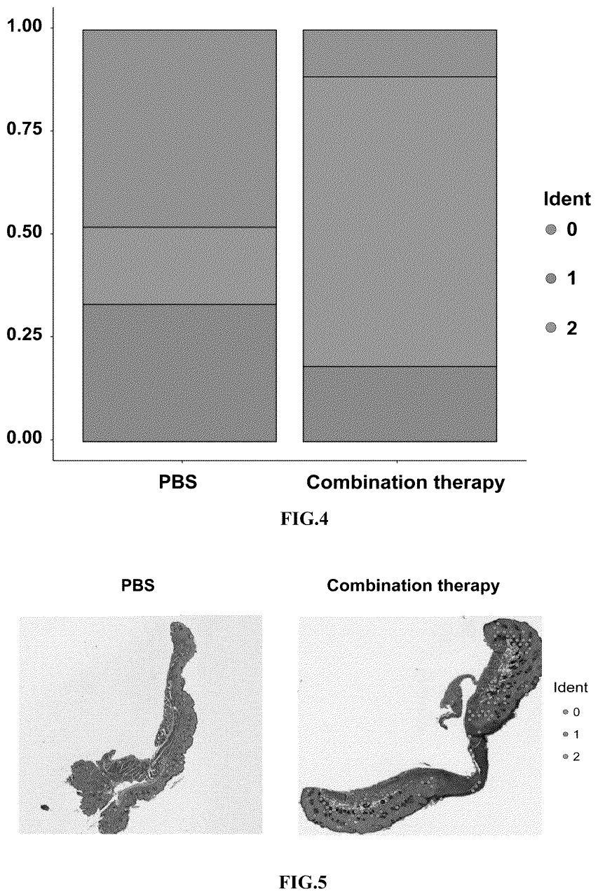

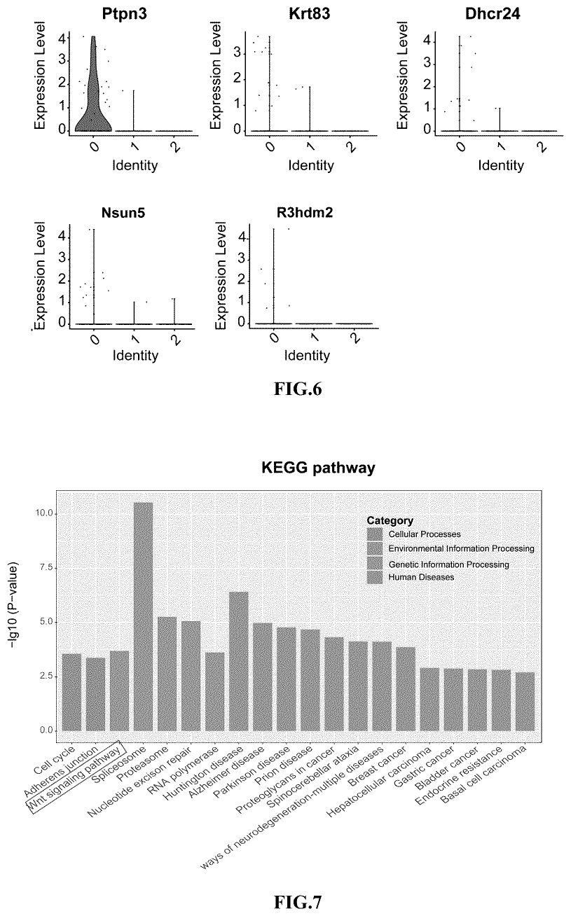

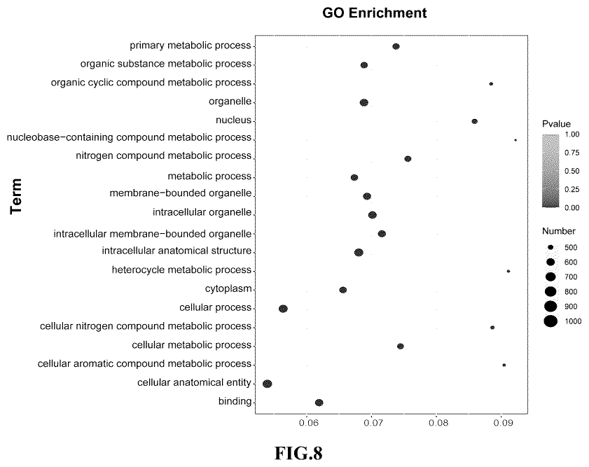

The analysis method of the spatial transcriptome-based 4D-printed stem cell scaffold for improving diabetic skin injury is based on the dorsal wound healing process of diabetic mice, the specific process is as follows:
first, the tissues were gathered, where the tissue of the PBS group was the skin tissue of the diabetic mice, and the tissue of the combined treatment group was 4D-printed stem cell scaffold+adipose mesenchymal stem cells+piRNA-hsa-32182 inhibitors;
the spatial transcriptome sequencing was performed by 10× Genomics spatial transcriptome sequencing analysis on the tissues, and two sets of data were obtained;
then, the Uniform Manifold Approximation and Projection (UMAP) method was used to cluster the meaningful cell subsets (such as T cells) into 3 different subsets, namely cluster 0, cluster 1 and cluster 2;
cell annotation was performed by reference (Haensel D, Jin S, Sun P, Cinco R, et al. Defining Epidermal Basal Cell States during Skin Homeostasis and Wound Healing Using Single-Cell Transcriptomics. Cell Rep 2020 Mar. 17; p30 (11): 3932-3947.e6.PMID: 32187560) and singR method, 8 main cell types including T cells, endothelial cells, fibroblasts, keratinocytes, macrophages, myofibroblasts, neutrophils, and smooth muscle cells were identified;
the spatial localization of celltypes in the two groups were visualized, the results were visualized in FIG. 1, and the proportions of various celltypes were counted and quantified, the results were shown in FIG. 2, it can be seen that the number distribution of cell types in the two groups had changed significantly, especially myofibroblasts, smooth muscle cells, macrophages, neutrophils and T cells, the synergistic effect of the above cell types ensured the effective progress of the wound healing process, thereby restoring the function and integrity of the tissue;
through the difference genes analysis of the Seurat package of R, the TOP 10 genes of different cell types before and after treatment were shown in FIG. 3, which revealed the changes in microenvironment before and after treatment;
finally, cluster 0, cluster 1, and cluster 2 in T cell located at the spatial level respectively were shown in FIG. 4, the proportion of cell subsets was quantified in FIG. 5. The number of Cluster 0 in T cells increased significantly in the combined treatment group, indicating that it played an active role in wound healing;
the gene expression profile of cluster 0 in T cells was analyzed by Seurat, the results were shown in FIG. 6, the TOP 5 significant differential genes were Ptpn3, Krt83, Dhcr24, Nsun5, and R3hdm2. Based on the expression characteristics, we name cluster 0 as Ptpn3-T cells;
the Ptpn3-T cells were analyzed by ClusterProfiler, and the healing mechanism was verified by GO analysis (gene function analysis) in FIG. 7 and KEGG analysis (Kyoto Encyclopedia of Genes and Genomes) in FIG. 8. The Ptpn3-T cells were significantly associated with Wnt signaling pathway.

The analysis method of a spatial transcriptome-based 4D-printed stem cell scaffold for improving diabetic skin injury involved in this example first identified Ptpn3-T cells related to wound healing, and speculated that the role of this cell in promoting healing activated the Wnt pathway, it provides new insights in diabetic wound healing and provides potential targets for future treatment strategies.

What is claimed is:

1. An analysis method of a spatial transcriptome-based 4D-printed stem cell scaffold for improving diabetic skin injury, comprising the following specific process:
firstly, the spatial transcriptome sequencing is performed on the tissues of the PBS treatment group and the combined treatment group, respectively;
then, cell clustering and cell annotation are performed, after identifying the main cell types, the spatial location of the two groups are visualized, and the proportions of various cell types are statistically quantified and plotted;
finally, the cell subsets with significant differential genes are enriched and verified.

2. The analysis method of the spatial transcriptome-based 4D-printed stem cell scaffold for improving diabetic skin injury according to claim 1, wherein the tissue of the PBS group is the skin tissue of diabetic mice, and the tissue of the combined treatment group is 4D-printed stem cell scaffold+adipose mesenchymal stem cells+piRNA-hsa-32182 inhibitors.

3. The analysis method of the spatial transcriptome-based 4D-printed stem cell scaffold for improving diabetic skin injury according to claim 1, wherein cell clustering is performed through UMAP.

4. The analysis method of the spatial transcriptome-based 4D-printed stem cell scaffold for improving diabetic skin injury according to claim 1, wherein cell annotation is performed by reference and singR method to identify the main cell types.

5. The analysis method of the spatial transcriptome-based 4D-printed stem cell scaffold for improving diabetic skin injury according to claim 1, wherein differential analysis is performed by Seurat package of R language to screen TOP 10 differential genes of different cell types before and after treatment.

6. The analysis method of the spatial transcriptome-based 4D-printed stem cell carrier for improving diabetic skin injury according to claim 4, wherein the reference is Haensel D, Jin S, Sun P. Cinco R et al. Defining Epidermal Basal Cell States during Skin Homeostasis and Wound Healing Using Single-Cell Transcriptomics. Cell Rep 2020 March 17; 30 (11): 3932-3947.e6. PMID: 32187560.

7. The analysis method of the spatial transcriptome-based 4D-printed stem cell scaffold for improving diabetic skin injury according to claim 1 wherein T cells with high expression of Ptpn3 related to injury healing are identified, the cell's role in promoting healing activates the Wnt pathway.

8. The analysis method of the spatial transcriptome-based 4D-printed stem cell scaffold for improving diabetic skin injury according to claim 2 wherein T cells with high expression of Ptpn3 related to injury healing are identified, the cell's role in promoting healing activates the Wnt pathway.

9. The analysis method of the spatial transcriptome-based 4D-printed stem cell scaffold for improving diabetic skin injury according to claim 3 wherein T cells with high expression of Ptpn3 related to injury healing are identified, the cell's role in promoting healing activates the Wnt pathway.

10. The analysis method of the spatial transcriptome-based 4D-printed stem cell scaffold for improving diabetic skin injury according to claim 4 wherein T cells with high expression of Ptpn3 related to injury healing are identified, the cell's role in promoting healing activates the Wnt pathway.

11. The analysis method of the spatial transcriptome-based 4D-printed stem cell scaffold for improving diabetic skin injury according to claim 5 wherein T cells with high expression of Ptpn3 related to injury healing are identified, the cell's role in promoting healing activates the Wnt pathway.

12. The analysis method of the spatial transcriptome-based 4D-printed stem cell scaffold for improving diabetic skin injury according to claim 6 wherein T cells with high expression of Ptpn3 related to injury healing are identified, the cell's role in promoting healing activates the Wnt pathway.
